(19) United States
(12) Patent Application Publication
(10) Pub. No.: US 2010/0279878 A1
(43) Pub. Date: Nov. 4, 2010

(54) Biomarkers for Septic Shock Patients

(76) Inventor: Hector R. Wong, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 12/599,444

(22) PCT Filed: May 14, 2008
(86) PCT No.: PCT/US08/06172

§ 371 Date: Jul. 1, 2010

Related U.S. Application Data
(60) Provisional application No. 60/917,716, filed on May 14, 2007.

(51) Int. Cl. C40B 30/00; G01N 33/53; G01N 33/50; C12Q 1/68; C07H 21/00

(52) U.S. Cl. 506/7; 435/7.1; 436/86; 436/501; 435/7.9; 435/6; 536/24.3

(57) ABSTRACT

The instant invention relates generally to the use of IL-8 as a biomarker in septic shock patients as an indicator of the likelihood of survival. The instant invention further relates to the use of IL-8 as a biomarker in septic shock patients for the selection of appropriate therapies. The instant invention further relates to the use of IL-8 as a biomarker for the purposes of structuring, conducting, or evaluating clinical trials or data from clinical trials.

### CROSS-REFERENCE TO RELATED APPLICATIONS

This application claims the benefit of U.S. Provisional Patent Application Ser. No. 60/917,716, filed May 14, 2007, which application is hereby incorporated by reference in its entirety and for all purposes whatsoever.

### BACKGROUND OF THE INVENTION

Septic shock is the leading cause of morbidity and mortality in the intensive care units. Despite increased knowledge about the pathophysiology underlying the clinical symptoms mortality remains high and has not decreased substantially over the last decades.

There are several causes of septic shock including bacterial, fungal and viral infections as well as noninvasive stimuli such as multiple trauma, severe burns, organ transplantations and pancreatitis. The fatal outcome of septic shock has recently been linked to the systemic release of substantial amounts of various cytokines in the body.

Septic shock requires prompt treatment since the patient's condition often deteriorates rapidly. Symptoms of septic shock include fever, hypothermia, falling blood pressure, rapid breathing, rapid heartbeat, skin lesions and leakage of plasma proteins into the tissues, metabolic acidosis and elevated plasma lactate. Septic shock is particularly characterized by maldistribution of blood flow and disturbances in tissue oxygen in various organs of the body. Distribution of blood flow may become heterogenous with subsequent under- and overperfusion of various tissues. These disturbances have been noted both at the macro- as well as at the microcirculatory level. Septic patient usually die as a result of poor tissue perfusion and injury followed by multiple organ failure.

Pediatric septic shock continues to be an important public health problem despite potent antibiotics and the development of pediatric intensive care units (34). There are approximately 42,000 cases per year of pediatric septic shock in the United States, with a mortality rate of approximately 10%, and higher mortality rates in children with co-morbidities such as cancer and prematurity (39). A great deal of basic research efforts have focused on the biological processes that occur in septic shock. While highly informative, a relative paucity of this information has been readily translated to the bedside in the form of meaningful therapeutic advances for children (12, 34). For example, multiple trials focused on immune modulation strategies have been conducted in adults with septic shock (1). Despite strong preclinical data, as well as strong phase I and II data, the majority of these strategies have failed when subjected to large scale, randomized placebo-controlled trials. Consequently, the majority of these strategies have not been effectively tested in the pediatric population. One notable exception is activated protein C, which recently received Food and Drug Administration approval for use in adults with septic shock (2, 4). Unfortunately, a phase III trial of activated protein C in children with septic shock was recently terminated early due to lack of efficacy (14). Current care for pediatric septic shock remains fundamentally based on antibiotics and supportive care (7, 34).

Therapeutic drug trials in pediatric septic shock have been universal failures to date. The most recent and notable example is that of activated protein C (APC). APC was recently approved by the FDA as the only drug specifically labeled for septic shock in adults. A phase III trial of APC in children was recently terminated at interim analysis secondary to lack of efficacy and a trend toward increased complications. There is a well founded perception that a primary reason why the pediatric APC trial failed was because many of the enrolled patients were destined to do well with standard care (i.e. they were not “sick” enough). Thus, when patients are enrolled into a drug trial having significant risks (hemorrhage), and they have a high likelihood of doing well with standard care, the risk to benefit ratio is negatively impacted for the overall patient cohort. Thus, there is a need for more effective stratification of patients at the time of enrollment.

Further, with respect to currently utilized and emerging high risk therapies, there is a need to differentiate septic shock patients who will respond to standard care from those who may require more aggressive, but higher risk therapies. There is further a need to identify such patients earlier in time, such that treatment may be administered as soon as possible.

### BRIEF SUMMARY OF THE INVENTION

The instant invention relates generally to the use of IL-8 as a biomarker in septic shock patients as an indicator of the likelihood of survival.

The instant invention further relates to the use of IL-8 as a biomarker in septic shock patients for the selection of appropriate therapies.

The instant invention further relates to the use of IL-8 as a biomarker for the purposes of structuring, conducting, or evaluating clinical trials or data from clinical trials.

In one aspect, the instant invention relates to the use of IL-8 as a biomarker for patient stratification for purposes of structuring, conducting, or evaluating clinical trials for the therapies for septic shock, comprising the steps of identifying an individual diagnosed with septic shock; determining IL-8 expression or protein level; comparing the determined IL-8 expression or protein level to a set of predetermined values for IL-8; then categorizing the individual for purposes of structuring, conducting or evaluating the clinical trial. In one embodiment, the described method may be retroactively applied to data derived from a clinical data for septic shock, wherein patients are stratified for the purposes of restructuring or removing patient data.

In another aspect, the instant invention relates to the use of IL-8 as a biomarker for patient stratification with respect to the treatment of septic shock, comprising the steps of identifying an individual diagnosed with septic shock; determining IL-8 expression or protein level; comparing the determined IL-8 expression or protein level to a set of predetermined values for IL-8; followed by classifying the patient as either appropriate or not appropriate for standard care.

In yet another aspect, the various embodiments of the instant invention relate to the use of IL-8 as a biomarker in pediatric patients diagnosed with septic shock, wherein the biomarker is used for patient stratification in structuring, conducting, or evaluating clinical trials for therapies for septic shock. The various embodiments relate further to the use of IL-8 as a biomarker in pediatric patients diagnosed with septic shock, wherein the biomarker is used for patient stratification for purposes of determining the appropriate treatment for septic shock.

In another embodiment, serum IL-8 may be used as a biomarker for stratifying patients for the purpose of the structuring, conducting, or evaluating of conducting therapeutic clinical trials focused on septic shock, particularly pediatric septic shock. For example, serum IL-8 levels may be used as a biomarker for exclusion of patients from such clinical trials. In this embodiment, a statistically derived negative or positive predictive value may be derived according to the methods described herein and used to stratify a patient population. In one embodiment, an interventional trial in septic shock uses a serum IL-8 level of less than about 200 pg/ml, 205 pg/ml, 210 pg/ml, 215 pg/ml, 220 pg/ml, 225 pg/ml, 230 pg/ml, 235 pg/ml, 240 pg/ml, 245 pg/ml or 250 pg/ml, to exclude patients that would otherwise be included based on clinical criteria.

In another embodiment, an interventional trial in septic shock uses a serum IL-8 level of less than about 200 pg/ml, 210 pg/ml, 220 pg/ml, 220 pg/ml, 240 pg/ml, 250 pg/ml, 260 pg/ml, 270 pg/ml, 280 pg/ml, 290 pg/ml or 300 pg/ml, to exclude patients that would otherwise be included based on clinical criteria.

In another embodiment, an interventional trial in septic shock uses a serum IL-8 level of less than about 300 pg/ml, 350 pg/ml, 400 pg/ml, 450 pg/ml, 500 pg/ml, 600 pg/ml, 700 pg/ml, 800 pg/ml, 900 pg/ml or less than about 1000 pg/ml, to exclude patients that would otherwise be included based on clinical criteria. This strategy would allow the trial to generate a more favorable risk to benefit ratio, and thus a higher likelihood of adequately assessing efficacy. This strategy may also be employed in post-hoc analysis of clinical trials, whereby data from subjects having IL-8 levels in a range described above, are eliminated from the data set.

In a yet further aspect, the instant invention relates to methods for the stratification of patients for purposes of structuring, conducting, or evaluating clinical trials for the therapies for septic shock, comprising the steps of identifying an individual suspected of having septic shock, obtaining a biological sample from the individual, determining the levels of IL-8 in the biological sample, then identifying the individual or data from an individual as appropriate for a clinical trial, wherein the individual or data from such individual having levels of IL-8 less than about 1000 pg/ml, or less than about 500 pg/ml or less than about 300 pg/ml or less than about 220 pg/ml, is excluded from the clinical trial.

In other embodiments, the instant invention relates to methods for the stratification of patients for determining the effective course of treatment, comprising the steps of identifying an individual suspected of having septic shock, obtaining a biological sample from the individual, determining the levels of IL-8 protein in the biological sample, then identifying the individual as appropriate for a particular treatment, wherein individuals having levels of IL-8 protein less than about 220 pg/ml, are considered appropriate candidates for standard care. Conversely, individuals having IL-8 levels greater than about 1000 pg/ml, or greater than about 1500 pg/ml may be considered as candidates for active agents that have been identified as efficacious for the treatment of septic shock in trials using IL-8 levels as exclusion criteria, or for other higher risk therapies. Individuals having IL-8 protein levels of less than about 1000 pg/ml, or less than about 500 pg/ml or less than about 300 pg/ml may be considered appropriate candidates for standard care, at the judgment of the treating physician or person conducting, structuring, or evaluating a clinical trial in accordance with the methods and disclosure herein.

In particular, the instant invention relates to point of care testing wherein IL-8 protein or expression is detected, and diagnostic tools embodying the same, wherein individuals can be stratified for purposes of treatment, or for purposes of structuring, conducting, or evaluating a clinical trial for the treatment of septic shock.

In other embodiments, the instant invention relates to the use of IL8 as a biomarker for the purposes described above, wherein mRNA levels of IL8 are determined and used to stratify patients for treatment or for purposes of structuring, conducting, or evaluating a clinical trial for the treatment of septic shock.

### BRIEF DESCRIPTION OF THE DRAWING

FIG. 1: Receiver operating curve (ROC) used for derivation of IL-8 cutoff >220 pg/ml having a 75% specificity and a 75% sensitivity for predicting 28 day mortality in children with septic shock. The patient cohort used to generate the ROC consists of 40 patients with septic shock (8 nonsurvivors) as previously reported (6). Area under the curve=0.857; 95% confidence interval=0.676 to 0.999; standard error=0.093; p value=0.002.

FIG. 2: Contingency table depicting sensitivity, specificity, positive predictive value (PPV), negative predictive value (NPV), for 28 day mortality, of IL-8 cutoff (220 pg/ml) when applied to validation data set #1 (n=139 patients). Also shown are the mortality likelihood ratios (LR) for a positive test (IL-8>220 pg/ml) and a negative test (IL-8≦220 pg/ml), respectively. Note that an IL8 serum level ≦220 pg/ml has a negative predictive value of 95% for 28 day mortality when applied to validation data set #1, with a confidence interval (CI) of 87% to 98%.

FIG. 3: Contingency table depicting sensitivity, specificity, positive predictive value (PPV), negative predictive value (NPV), for 28 day mortality, of IL-8 cutoff (220 pg/ml) when prospectively applied to validation data set #2 (n=193). Also shown are the mortality likelihood ratios (LR) for a positive test (IL-8>220 pg/ml) and a negative test (IL-8≦220 pg/ml), respectively. Note that an IL8 serum level ≦220 pg/ml has a negative predictive value of 94% for 28 day mortality when prospectively applied to validation data set #2, with a confidence interval (CI) of 86% to 98%.

FIG. 4: Contingency table depicting sensitivity, specificity, positive predictive value (PPV), negative predictive value (NPV), for 28 day mortality, of IL-8 cutoff (220 pg/ml) when applied to the combined validation data sets #1 and #2 (n=332). Also shown are the mortality likelihood ratios (LR) for a positive test (IL-8>220 pg/ml) and a negative test (IL-8≦220 pg/ml), respectively. Note that an IL8 serum level ≦220 pg/ml has a negative predictive value of 95% for 28 day mortality when applied to combined validation data sets #1 and #2, with a confidence interval (CI) of 90% to 98%.

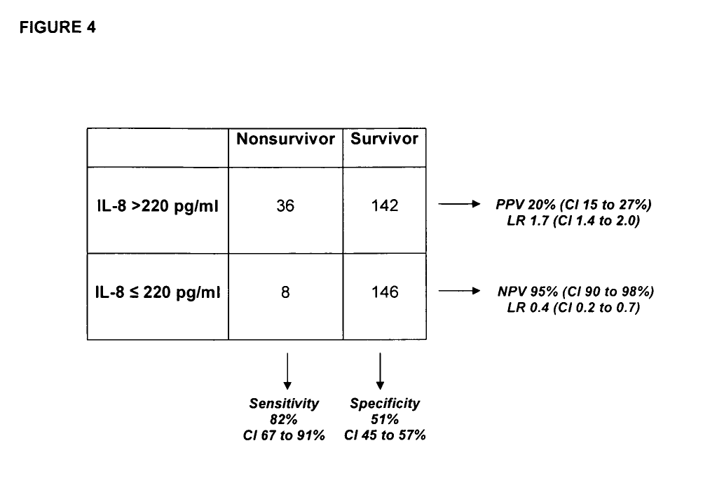

FIG. 5: An IL8 cutoff value of 220 pg/ml segregates children with septic shock into two significantly different risk groups for 28 day mortality (p<0.001, Chi-square). Data are derived from 332 patients after combining validation data sets #1 and #2.

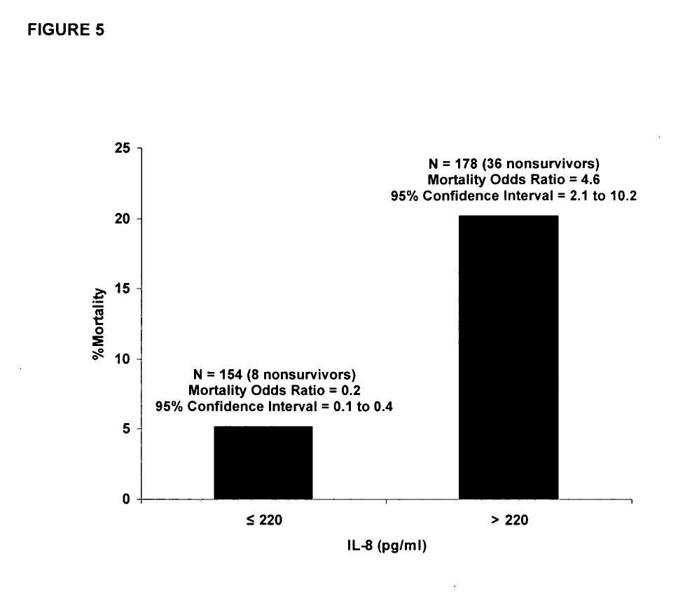

### DETAILED DESCRIPTION OF THE INVENTION

### Definitions

For convenience, certain terms employed in the specification, examples and claims are collected here. These definitions should be read in light of the remainder of the disclosure and understood as by a person of skill in the art. Unless defined otherwise, all technical and scientific terms used herein have the same meaning as commonly understood by a person of ordinary skill in the art. Although any methods and materials similar or equivalent to those described herein can be used in the practice or testing of the present invention, the preferred methods, devices, and materials are now described. All references, publications, patents, patent applications, and commercial materials mentioned herein are incorporated herein by reference for the purpose of describing and disclosing the materials and/or methodologies which are reported in the publications which might be used in connection with the invention. Nothing herein is to be construed as an admission that the invention is not entitled to antedate such disclosure by virtue of prior invention. In order to provide a clear and consistent understanding of the specification and claims, including the scope to be given such terms, the following definitions are provided:

The articles “a” and “an” are used herein to refer to one or to more than one (i.e., to at least one) of the grammatical object of the article. By way of example, “an element” means one element or more than one element.

A “biological sample” encompasses any sample obtained from a living system or subject. The definition encompasses blood, serum, tissue, and other samples of biological origin that can be collected from a living system, subject or individual. Preferably, biological samples are obtained through sampling by minimally invasive or non-invasive approaches (e.g., urine collection, stool collection, blood drawing, needle aspiration, and other procedures involving minimal risk, discomfort or effort). Biological samples can be gaseous (e.g., exhaled breath). Biological samples are often liquid (sometimes referred to as a “biological fluid”). Liquid biological samples include, but are not limited to, urine, blood, interstitial fluid, edema fluid, saliva, lacrimal fluid, inflammatory exudates, synovial fluid, abscess, empyema or other infected fluid, cerebrospinal fluid, sweat, pulmonary secretions (sputum), seminal fluid, feces, bile, intestinal secretions, and others. Biological samples include samples that have been manipulated in any way after their procurement, such as by treatment with reagents, solubilization, or enrichment for certain components, such as proteins or polynucleotides. The term “biological sample” also encompasses a clinical sample such as serum, plasma, other biological fluid, or tissue samples, and also includes cells in culture, cell supernatants and cell lysates.

As used herein, the term “biomarker” refers to a physical, biochemical, or physiologic measurement from or on the organism that represents a true or intended mechanistic target of a compound or a mechanistic event believed to be responsible for, or contributing in, a causal manner to the initiation, progression, severity, pathology, aggressiveness, grade, activity, disability, mortality, morbidity, disease sub-classification or other underlying pathogenic or pathologic feature of one or more diseases. A biomarker may be the target for monitoring the outcome of a therapeutic intervention (i.e., the functional or structural target of a drug agent). “Biomarker” refers to biochemical processes that are involved in, or are believed to be involved in, the etiology or progression of a disease or disorder. The biochemical process (i.e., the flow of molecules through a targeted metabolic pathway or network) is the focus of analysis (as disclosed herein) since it is the underlying changes of the biochemical process (i.e., molecular flux rates) that may be the significant or authentic target for treatment or diagnostic monitoring of the disease or disorder.

The terms “drug,” “pharmaceutically active agent,” “bioactive agent,” “therapeutic agent,” and “active agent” may be used interchangeably and refer to a substance, such as a chemical compound or complex, that has a measurable beneficial physiological effect on the body, such as a therapeutic effect in treatment of a disease or disorder, when administered in an effective amount. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, prodrugs, active metabolites, isomers, fragments, analogs, solvates hydrates, radioisotopes, etc.

“Higher risk” or “aggressive” therapy as used herein will be understood by one of ordinary skill in the art and includes, for example, plasmapheresis, high dose ultrafiltration, extracorporeal membrane oxygenation. Such therapies are also intended to include newly developed therapies (e.g., active agents or invasive procedures) considered to be higher risk therapies, and active agents that are considered higher risk by one of skill in the art. To this end, Carcilllo, et al, “Clinical practice parameters for hemodynamic support of pediatric and neonatal patients in septic shock,” Crit. Care Med. 2002 June; 30(6):1365-78, Dellinger R P et al., “Surviving Sepsis Campaign guidelines for management of severe sepsis and septic shock,” Crit. Care Med. 2004 March; 32(3):858-73. and Annane D. et al, “Septic Shock,” Lancet, 2005 Jan. 1-7; 365(9453):63-68 are incorporated herein by reference.

An “individual” is a vertebrate, preferably a mammal, more preferably a human.

The term “prophylactic” or “therapeutic” treatment is art-recognized and refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The phrase “standard care” with respect to septic shock is known to one of ordinary skill in the art and generally includes antibiotics and organ support.

The phrase “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the individual and disease condition being treated, the weight and age of the individual, the severity of the disease condition, the manner of administration and the like, which can readily be, determined by one of ordinary skill in the art.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

The practice of the present invention will employ, unless otherwise indicated, conventional techniques of molecular biology (including recombinant techniques), microbiology, cell biology and biochemistry, which are within the skill of the art.

We have been addressing the problem of pediatric septic shock through a translational research approach that leverages the power of microarray technology and bioinformatics. Over the last 4 years we have organized a national-level research network that has allowed us to develop a genomic databank of children with septic shock. The databank now contains whole blood derived RNA, DMA, and serum from over 250 subjects. As an initial approach to mining this databank, we have conducted genome level expression profiles by way of microarray technology and have recently published our first manuscript in this area (Wong et al. Physiol Genomics. 2007 Mar. 20 [Epub ahead of print]). The first approximation of the genome level expression profiles of pediatric septic shock indicates that this is a powerful and feasible approach for discovering novel therapeutic targets and biomarkers.

The data presented herein were recently published in Wong et al. Physiol Genomics. 2007 Mar. 20 attached, and incorporated herein by reference. The data represent the “Day 1” (i.e. within 24 hours of admission to the pediatric intensive care unit) genome-level expression profiles of 42 children with septic shock relative to 15 normal control children.

### Interleukin-8 as a Tool for Patient Stratification

Interleukin-8 (IL-8) is a 69-79 amino-acid cytokine produced by various cell types including neutrophils, endothelium, microglia and astrocytes. IL-8 is now referred to as CXCL8. In fact, of the more than 16,000 publications on chemokines in the last two and half decades, nearly 7,000 of these publications have cited IL-8/CXCL8. IL-8/CXCL8 is a member of the CXC chemokine family that is also classified by whether a member contains the three-amino acid sequence of glutamic acid-leucine-arginine (Glu-Leu-Arg, “ELR” motif) that immediately precedes the first cysteine amino acid residue in the primary structure of the protein. The members of the CXC chemokine family that contain this motif are referred to as ELR+CXC chemokines and have their primary biological effect in promoting neutrophil recruitment and angiogenesis. The gene for IL-8/CXCL8 is found on human chromosome 4, q12-21, and consists of four exons and three introns. The 5′-flanking region of IL-8/CXCL8 contains the usual “CCAAT” and “TATA” box-like structures. In addition, this region has a number of potential binding sites for several nuclear factors. The IL-8/CXCL8 promoter region is regulated in a cell-specific fashion requiring a NF-κB element plus either activator protein (AP)-1 or a C/EBP (NF-IL-6) element under conditions of transcriptional induction with tumor necrosis factor (TNF)- or IL-1. Although the IL-8/CXCL8 promoter has two C/EBP (5′ and 3′) cis elements with NF-κB nested between them, the 5′-C/EBP element appears to be the only C/EBP element involved in transcriptional regulation of IL-8/CXCL8. These features of IL-8 are described in Strieter, R., “Interleukin-8: a very important chemokine of the human airway epithelium,” Am J Physiol Lung Cell Mol Physiol 283: L688-L689, 2002.

In one aspect of the invention, IL-8 protein or expression may be used as a biomarker for the stratification of patients for purposes of structuring, conducting, or evaluating clinical trials. This includes, but is not limited to, using IL-8 levels to define a patient's entry into or exclusion from a clinical trial; defining a particular patient as a control or test subject; conducting post-hoc evaluation of clinical trial data; or other uses related to structuring the clinical trial or resulting data.

In another embodiment, the invention relates to the use of IL-8 protein or expression as a biomarker for the stratification of patients for purposes of selecting the appropriate treatment for septic shock, wherein patients diagnosed with septic shock can be characterized as either appropriate candidates for standard care or higher risk therapies.

In the genome level expression profiles of pediatric septic shock patients, we found 63 gene probes that were differentially regulated between the survivors and nonsurvivors in this patient cohort. One of these, interleukin-8 (IL-8) was highly expressed among the nonsurvivors. Because IL-8 protein can be readily measured in serum, we chose this particular gene for validation of our microarray data and found that, indeed, nonsurvivors had significantly higher serum levels of IL-8 protein compared to survivors (Wong et al. Physiol Genomics. 2007 Mar. 20 [Epub ahead of print]).

As such, in one aspect of the instant invention is provided a method for the stratification of patients diagnosed with septic shock comprising the steps of: determining the level of IL-8 protein or expression; comparing the IL-8 level to a set of predetermined values as described herein; and making a determination as to whether the patient or patient data should be excluded from a trial as a test subject. In this aspect of the instant invention, for example, a protein or expression level of IL-8 correlating to a negative predictive value for death of about 80-100%, or about 90-95% or about 95% indicates that the patient or patient data should be excluded from a clinical trial. In yet a further aspect, IL-8 protein levels may be used wherein a cut-off of less than about 1000 ng/ml, or about 500 ng/ml, or about 300 ng/ml, or about 220 ng/ml is a suitable cutoff value for determining whether a patient or patient data should be excluded from a clinical trial.

In an additional aspect of the instant invention is provided a method for the stratification of patients diagnosed with septic shock comprising the steps of: determining the level of IL-8 protein or expression; comparing the IL-8 level to a set of predetermined values as described herein; and making a determination as to whether the patient is appropriate for standard care or for higher risk, more aggressive treatment. In this aspect of the instant invention, for example, a protein or expression level of IL-8 correlating to negative predictive value for death of about 80-100%, or about 90-95% or about 95% indicates that the patient is an appropriate subject for standard care. In yet a further aspect, an IL-8 protein level cutoff of less than about 1000 ng/ml, or less than about 500 ng/ml, or less than about 300 ng/ml, or less than about 220 ng/ml is a suitable cutoff value for determining whether a patient is appropriate for standard care. In this aspect of the invention, these methods may be used to determine whether a patient is likely to proceed to septic shock, such that treatment for septic shock may be administered as soon as possible.

In follow-up to this finding, we next generated a receiver operating curve (unpublished data) and derived a serum IL-8 level >220 pg/ml, within 24 hours of admission to the pediatric intensive care unit, as having a sensitivity and specificity of 75% for predicting death based on this training data set of 40 patients. We next applied this cutoff, prospectively, to a validation data set of 99 patients with septic shock (10 nonsurvivors) and found that 8 of 10 nonsurvivors had a serum IL-8 level >220 pg/ml (80% sensitivity) and 55 of 89 survivors had a serum IL-8 level <220 pg/ml (62% specificity).

We next combined both the training data set and validation data set (139 total patients with 18 nonsurvivors) and derived the following observations regarding a serum IL-8 level of 220 pg/ml:

A serum IL-8 level >220 pg/ml has a sensitivity of 78% and a specificity of 64% for predicting death in children with septic shock.

A serum IL-8 level >220 pg/ml has a positive predictive value of 25% for death.

A serum IL-8 level <220 pg/ml has a negative predictive value of 95% for death.

The sensitivity, specificity, and positive predictive value of IL-8, for this particular study, turn out to not be particularly robust. The negative predictive value, however, is quite robust: an IL-8 level <220 pg/ml predicted survival in the context of septic shock with 95% certainty.

While a value of less than about 220 pg/ml IL-8 has a negative predictive value of 95% for death, other values may be used, as determined using standard methods as readily appreciated by one of skill in the art. That is, the quality of the differentiation between survivors and nonsurvivors having sepsis is judged by sensitivity, specificity, positive predictive value (PPV), and negative predicted value (NPV). The term “sensitivity” refers to the ability of a test to identify individuals who are truly positive for a test parameter. The measure of sensitivity is ratio of the number of subjects identified as positive by the test method divided by the total number of truly positive samples as determined by the reference method (i.e., the true status). The term “specificity” refers to the ability of a test to identify individuals who are truly negative for the test parameter. The measure of specificity is the ratio of the number of subjects identified as negative by the test method divided by the total number of truly negative subjects as determined by the reference method. The expression “positive predictive value” or “PPV” is the probability of being truly positive given a positive test result. It is the ratio of the number of truly positive subjects identified as positive by the test method divided by the total number of positive subjects as determined by the test method. The expression “negative predictive value” or “NPV” is the probability of being truly negative given a negative test result. With respect to the instant methods, this is the probability of a subject being appropriate for standard care. It is the ratio of the number of truly negative subjects identified by the test method divided by the total number of negative subjects as determined by the test method. The expression “p value” is a parameter that refers to the ability to separate two partially overlapping populations using the chi² test. The smaller the value of p, the better is the separation between the two populations.

Negative predictive value (NPV) can be calculated, for example, using the following formula:

$$NPV = \frac{number of true negatives}{Number of true negatives + number of false negatives}$$

Or, alternatively, NPV can be calculated using the following:

$$NPV = \frac{specificity \times ( 1 - prevalence )}{Specificity \times ( 1 - prevalence ) + ( 1 - sensitivity ) \times prevalence}$$

Specificity and sensitivity are terms of art and calculations thereof may be readily determined by one of ordinary skill in the art.

Using these calculations as applied to the instant study population, yields, for example values of less than about 1000 pg/ml, or less than about 500 pg/ml, or less than about 300 pg/ml that may be used for the instant invention to stratify patients having septic shock. One of skill in the art will readily appreciate that protein or expression levels of IL-8 and the corresponding negative or positive predictive values may be determined and may fluctuate with respect to the size and heterogeneity of a sample population. This aspect of the instant invention is not necessarily limited to a specific cutoff value, but to the use of IL-8 as a biomarker for patient stratification in general. Determination of the positive and negative predictive values with respect to IL-8 levels in accordance with the scope of the present invention is well within the scope of one of ordinary skill in the art.

Typically, in the clinical setting, with respect to predictive values, a 5% error is usually the upper limit of tolerance to declare something as significant. As such, in certain embodiments, at least a 95% PPV or NPV may be used as a basis for determining patient stratification in accordance with the methods disclosed herein. In other embodiments however, other statistically derived limits, for example, a PPV or NPV of about 90-94% or about 85-90% or about 80-85% may be used with the methods of the instant invention. The application of these values in stratifying patients is at the judgment of the treating clinician or the individual responsible for structuring, conducting, or evaluating a clinical trial.

Depending on the sample population, one of ordinary skill in the art, using the methods described herein, will be able to readily derive predictive values from additional sample populations and corresponding cut-off values for IL-8 (expression or protein levels). Expression or protein levels of a given amount will typically correlate with various predictive values. For example, the following IL-8 levels and corresponding negative predictive values for death were determined with respect to the study population described herein: an IL-8 level of <1000 pg/ml has a negative predictive value for death of about 92%, while an IL-8 level of <500 pg/ml has a negative predictive value for death of about 92%, and an IL-8 level of <300 pg/ml has a negative predictive value for death of about 90%. As such, these provide examples of calculated levels may be used as cutoff values for stratification of patients with septic shock for structuring, conducting, or evaluating clinical trials, or for stratification of patients for selection of an appropriate treatment.

We believe that this observation (the negative predictive value of IL-8) is very powerful for the following reasons. As discussed above, therapeutic drug trials in pediatric septic shock have been universal failures to date. There is a well founded perception that in the recent ARC trial, a primary reason why the pediatric trial failed was because many of the enrolled patients were destined to do well with standard care (i.e. they were not “sick” enough), thereby negatively impacting the risk to benefit ratio for the overall patient cohort. Thus, there is a need for more effective stratification of patients at the time of enrollment.

Accordingly, serum IL-8 may be used as a biomarker for stratifying patients for the purpose of the structuring, conducting, or evaluating of conducting therapeutic clinical trials focused on septic shock, particularly pediatric septic shock. For example, serum IL-8 levels may be used as a biomarker for exclusion of patients from such clinical trials. In this embodiment, a statistically derived negative or positive predictive value may be derived according to the methods described herein and used to stratify a patient population. In one embodiment, an interventional trial in septic shock uses a serum IL-8 level of less than about 200 pg/ml, 205 pg/ml, 210 pg/ml, 215 pg/ml, 220 pg/ml, 225 pg/ml, 230 pg/ml, 235 pg/ml, 240 pg/ml, 245 pg/ml or 250 pg/ml, to exclude patients that would otherwise be included based on clinical criteria.

In another embodiment, an interventional trial in septic shock uses a serum IL-8 level of less than about 200 pg/ml, 210 pg/ml, 220 pg/ml, 220 pg/ml, 240 pg/ml, 250 pg/ml, 260 pg/ml, 270 pg/ml, 280 pg/ml, 290 pg/ml or 300 pg/ml, to exclude patients that would otherwise be included based on clinical criteria.

In another embodiment, an interventional trial in septic shock uses a serum IL-8 level of less than about 300 pg/ml, 350 pg/ml, 400 pg/ml, 450 pg/ml, 500 pg/ml, 600 pg/ml, 700 pg/ml, 800 pg/ml, 900 pg/ml or less than about 1000 pg/ml, to exclude patients that would otherwise be included based on clinical criteria. This strategy would allow the trial to generate a more favorable risk to benefit ratio, and thus a higher likelihood of adequately assessing efficacy. This strategy may also be employed in post-hoc analysis of clinical trials, whereby data from subjects having IL-8 levels in a range described above, are eliminated from the data set.

In another embodiments, measurement of IL-8 or other high risk septic shock gene expression, or corresponding protein levels, may be used to separate those patients that are in need of drastic, higher risk treatments from those patients who are likely to improve with less invasive therapies, such as antibiotic treatment.

For example, a newborn with a high temperature must be evaluated for foci of severe infection. This evaluation can include invasive measures such as lumbar puncture in order to rule out meningitis. Often the febrile newborn requires hospitalization and treatment with broad spectrum antibiotics until a source of the fever has been determined. If a subgroup of the newborn population could be identified as having a greater probability of surviving the incidence of septic shock, diagnostic and therapeutic measures could be tailored to the degree of risk. Lumbar puncture could be restricted to the high-risk infant, for example. Brik, et al., “Evaluation of febrile infants under 3 months of age: is routine lumbar puncture warranted?” Isr. J. Med. Sci. 33(2):93-97, 1997. Or, for example, low risk infants could be managed as outpatients or discharged quickly from the hospital, offering an important cost-saving in this era of managed care. Durongpisitkul, et al., “The appropriateness of early discharge of hospitalized children with suspected sepsis,” J. Fam. Pract. 44(1):91-96, 1997. Infants, children or other subjects at particular risk for certain severe systemic infections could be treated with infection-specific agents, or could be treated earlier or more aggressively.

For example, serum levels below about 220 pg/mL have a 95% negative predictive value for death, and correlate to septic shock patients who are likely to benefit from standard care. Conversely, IL8 levels may be used to identify patients diagnosed with septic shock likely to benefit from more aggressive, higher risk therapies. Other predictive ranges for mRNA levels of IL-8 can also be used, and can be readily determined using standard methods that are well within the abilities of one with ordinary skill in the art as described above.

Measurements of protein or mRNA may be carried out using standard techniques known in the art. In one embodiment, the outcome of the measurement is obtained rapidly, within 24 hours or less, or about 3 hours, so that suitable therapies may be given immediately. Relatively rapid test measurements, such as dipsticks, test strips, chip technologies, tissue blots, or other methods can be used.

The various aspects of the instant invention as described herein may also be applied to individuals diagnosed with sepsis, or sepsis progressing to septic shock, or sepsis likely to progress to septic shock.

### Assays

Methods for determining the concentration of analytes in samples from individuals are well known in the art and readily adapted by the skilled person in the context of the present invention to determine the presence or amount of the protein markers or fragments thereof, or antibodies against the markers in a sample from a patient. The results of such assays can in turn allow a physician to determine whether a patient suffers from a condition or is at risk of developing acute rejection or an associated condition. It may also allow the physician to optimize the treatment of the conditions. Thus, this allows for planning of appropriate therapeutic and/or prophylactic treatment, permitting stream-lining of treatment by targeting those most likely to benefit.

The methods typically employ a biological sample from patient such as blood, serum, tissue, serum, urine or other suitable body fluids. A preferred patient sample is blood.

The assay methods for determining the concentration of the protein markers or antibodies typically employ binding agents having binding sites capable of specifically binding to protein markers, or fragments thereof, or antibodies in preference to other molecules. Examples of binding agents include antibodies, receptors and other molecules capable of specifically binding the analyte of interest. Conveniently, the binding agents are immobilized on solid support, e.g. at defined, spatially separated locations, to make them easy to manipulate during the assay.

The sample is generally contacted with the binding agent(s) under appropriate conditions which allow the analyte in the sample to bind to the binding agent(s). The fractional occupancy of the binding sites of the binding agent(s) can then be determined either by directly or indirectly labeling the analyte or by using a developing agent or agents to arrive at an indication of the presence or amount of the analyte in the sample. Typically, the developing agents are directly or indirectly labeled (e.g. with radioactive, fluorescent or enzyme labels, such as horseradish peroxidase) so that they can be detected using techniques well known in the art. Directly labeled developing agents have a label associated with or coupled to the agent. Indirectly labeled developing agents may be capable of binding to a labeled species (e.g. a labeled antibody capable of binding to the developing agent) or may act on a further species to produce a detectable result. Thus, radioactive labels can be detected using a scintillation counter or other radiation counting device, fluorescent labels using a laser and confocal microscope, and enzyme labels by the action of an enzyme label on a substrate, typically to produce a color change. In further embodiments, the developing agent or analyte is tagged to allow its detection, e.g. linked to a nucleotide sequence which can be amplified in a PCR reaction to detect the analyte. Other labels are known to those skilled in the art are discussed below. The developing agent(s) can be used in a competitive method in which the developing agent competes with the analyte for occupied binding sites of the binding agent, or non-competitive method, in which the labeled developing agent binds analyte bound by the binding agent or to occupied binding sites. Both methods provide an indication of the number of the binding sites occupied by the analyte, and hence the concentration of the analyte in the sample, e.g. by comparison with standards obtained using samples containing known concentrations of the analyte.

In alternative embodiments, the analyte can be tagged before applying it to the support comprising the binding agent. In a preferred format, the presence or amount of a marker set out in Table 2, or antibodies against these antigens, is determined in an ELISA assay.

There is also an increasing tendency in the diagnostic field towards miniaturization of such assays, e.g. making use of binding agents (such as antibodies or nucleic acid sequences) immobilized in small, discrete locations (microspots) and/or as arrays on solid supports or on diagnostic chips. These approaches can be particularly valuable as they can provide great sensitivity (particularly through the use of fluorescent labeled reagents), require only very small amounts of biological sample from individuals being tested and allow a variety of separate assays can be carried out simultaneously. This latter advantage can be useful as it provides an assay employing a plurality of analytes to be carried out using a single sample. Examples of techniques enabling this miniaturized technology are provided in WO84/01031, WO88/01058, WO89/01157, WO93/08472, WO95/18376, WO95/18377, WO95/24649 and EP 0 373 203 A. Thus, in a further aspect, the present invention provides a kit comprising a support or diagnostic chip having immobilized thereon a plurality of binding agents capable of specifically binding different protein markers or antibodies, optionally in combination with other reagents (such as labeled developing reagents) needed to carrying out an assay. In this connection, the support may include binding agents specific for analytes such as vimentin, e.g. as disclosed in U.S. Pat. No. 5,716,787.

### Expression of Proteins

Following the identification of the protein markers associated with acute rejection, large amounts of the protein may be produced using expression techniques well known in the art. The protein produced in this way may be used as a binding agent, immobilizing it on solid support in an assay for antibodies in a sample from a patient, or as an immunogen to produce antibodies. Alternatively, the protein, or fragments thereof, may be used in the therapeutic treatment of organ transplant rejection, i.e. to ameliorate the deleterious effect of the antibodies.

Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others. A common, preferred bacterial host is E. coli.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

After transforming the host cells with the nucleic acid encoding the proteins, they an be produced by causing or allowing expression from the nucleic acid, e.g. by culturing host cells (which may include cells actually transformed although more likely the cells will be descendants of the transformed cells) under conditions for expression of the gene, so that the encoded polypeptide is produced. If the polypeptide is expressed coupled to an appropriate signal leader peptide it may be secreted from the cell into the culture medium. Following production by expression, a polypeptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers.

### Antibodies

In alternative embodiments of the invention, antibodies capable of binding the protein associated with acute rejection may be needed, e.g. for use in assays to determine the presence or amount of a given protein in a sample or for therapeutic use in reducing the deleterious effect of a protein in vivo. Thus, the present invention also provides the production of antibodies having the property of specifically binding to the marker proteins identified herein, or fragments or active portions thereof.

The production of monoclonal antibodies is well established in the art. Monoclonal antibodies can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP 0 184 187 A, GB 2 188 638 A or EP 0 239 400 A. A hybridoma producing a monoclonal antibody may be subject to genetic mutation or other changes, which may or may not alter the binding specificity of antibodies produced.

These antibodies may be specific in the sense of being able to distinguish between the polypeptide it is able to bind and other human polypeptides for which it has no or substantially no binding affinity (e.g. a binding affinity more than 10.sup.3, more preferably 10.sup.4 and more preferably 10.sup.5 times better than to unrelated molecules). Specific antibodies bind an epitope on the molecule which is either not present or is not accessible on other molecules. Antibodies are also useful in purifying the polypeptide or polypeptides to which they bind, e.g. following production by recombinant expression from encoding nucleic acid.

Preferred antibodies according to the invention are isolated, in the sense of being free from contaminants such as antibodies able to bind other polypeptides and/or free of serum components. Monoclonal antibodies are preferred for some purposes, though polyclonal antibodies are within the scope of the present invention.

Antibodies may be obtained using techniques which are standard in the art. Methods of producing antibodies include immunizing a mammal (e.g. mouse, rat, rabbit, horse, goat, sheep or monkey) with the protein or a fragment thereof. Antibodies may be obtained from immunized animals using any of a variety of techniques known in the art, and screened, preferably using binding of antibody to antigen of interest. For instance, Western blotting techniques or immunoprecipitation may be used (Armitage et al, Nature, 357:80 82, 1992). Isolation of antibodies and/or antibody-producing cells from an animal may be accompanied by a step of sacrificing the animal.

As an alternative or supplement to immunizing a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunized with any of the proteins (or fragments), or may be one constructed using sequences obtained from an organism which has been exposed to the antigen of interest.

Antibodies according to the present invention may be modified in a number of ways. Indeed the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope.

Hybridomas capable of producing antibody with desired binding characteristics are within the scope of the present invention, as are host cells, eukaryotic or prokaryotic, containing nucleic acid encoding antibodies (including antibody fragments) and capable of their expression. The invention also provides methods of production of the antibodies including growing a cell capable of producing the antibody under conditions in which the antibody is produced, and preferably secreted.

Antibodies for use in the assays described herein as binding or developing agents may be labeled. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule. One favored mode is by covalent linkage of each antibody with an individual fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine. Other reporters include macromolecular colloidal particles or particulate material such as latex beads that are colored, magnetic or paramagnetic, and biologically or chemically active agents that can directly or indirectly cause detectable signals to be visually observed, electronically detected or otherwise recorded. These molecules may be enzymes which catalyze reactions that develop or change colors or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed. Other techniques that can be sued to label antibodies include tagging, e.g. with a nucleotide sequence which can be amplified by PCR.

The mode of determining binding is not a feature of the present invention and those skilled in the art are able to choose a suitable mode according to their preference and general knowledge.

Antibodies according to the present invention may be used in screening for the presence of a polypeptide, for example in a test sample containing cells or cell lysate as discussed, and may be used in purifying and/or isolating a polypeptide according to the present invention, for instance following production of the polypeptide by expression from encoding nucleic acid therefore. Antibodies may modulate the activity of the polypeptide to which they bind and so, if that polypeptide has a deleterious effect in an individual, may be useful in a therapeutic context (which may include prophylaxis).

An antibody may be provided in a kit, which may include instructions for use of the antibody, e.g. in determining the presence of a particular substance in a test sample. One or more other reagents may be included, such as labeling molecules, buffer solutions, elutants and so on. Reagents may be provided within containers which protect them from the external environment, such as a sealed vial.

Specific alterations in inflammatory and insulin resistance cytokines (IL-1-alpha, IL-1.beta., IL-2, IL-4, TNF-alpha, TNF-R, IL-6, MCP-1, IL-8, IL-11, IL-12, and VCAM), and angiogenesis related growth factors (PlGF, FGF-2) and a growth factor antagonist (sFlt-1) can be used as secondary markers. In any event, these cytokines, chemokines, and angiogenesis related growth factors, or antagonists thereof, serve as early biomarkers for disease, and characterize subjects at high risk for future disease.

A “reference” can also be referred to as a “control” profile. A reference profile can be generated from a sample taken at a particular time point in the pregnancy of a normal individual or from an individual having a gestational disorder. The reference profile, or plurality of reference profiles, can be used to establish threshold values for the levels of, for example, specific cytokines in a sample. A “reference” profile includes a profile generated from one or more pregnant women having a gestational disorder or a profile generated from one or more pregnant women having a normal pregnancy.

A reference profile can be in the form of an array “signature” or “pattern” of specific identifiable biomarkers. The signature can also be described as a number or series of numbers that correspond to values attributed to the biomarkers identified by the array.

In addition to being expressed as a signature, a reference profile can be in the form of a threshold value or series of threshold values. For example, a single threshold value can be determined by averaging the values of a series of cytokine levels from pregnant women having normal pregnancies. Similarly, a single or two or more threshold values can be determined by averaging the values of a series of cytokine levels from pregnant women having a gestational disorder. Thus, a threshold value can have a single value or a plurality of values, each value representing a level of a specific cytokine or growth factor, or antagonist thereof, detected in a urine or blood sample, e.g., of a pregnant individual, or multiple individuals, having a gestational disorder.

### Methods

Patients: The study protocol was approved by the individual Institutional Review Boards of each participating institution. Children <10 years of age admitted to the pediatric intensive care unit (PICU) and meeting criteria for septic shock were eligible for the study. Septic shock was defined using pediatric-specific criteria (18). Control patients were recruited from the participating institutions using the following exclusion criteria: any acute illness, a recent febrile illness (within 2 weeks), recent use of anti-inflammatory medications (within 2 weeks), or any history of chronic or acute disease associated with inflammation.

Sample and data collection: After obtaining informed consent, blood samples (for RNA and serum isolation) were obtained within 24 hours of admission to the PICU, heretofore referred to as “Day 1” of septic shock. Severity of illness was calculated using the PRISM III score (27), and organ failure was defined using pediatric-specific criteria (18, 28, 40). Annotated clinical and laboratory data were collected daily while in the PICU. All study patients were followed for 28 days to determine survival. Clinical, laboratory, and biological data were entered and stored using a web-based database developed locally.

RNA extraction, microarray hybridization, and microarray analysis: The data and protocols described in this manuscript have been deposited in the NCBI Gene Expression Omnibus (GEO, http://www.ncbi.nlm.nih.gov/geo/) and are accessible through GEO Series accession number GSE4607.

Total RNA was isolated from whole blood samples using the PaxGene™ Blood RNA System (PreAnalytiX, Qiagen/Becton Dickinson, Calif.) according the manufacturer's specifications. Microarray hybridization was performed by the Affymetrix Gene Chip Core facility at Cincinnati Children's Hospital Research Foundation previously described using the Human Genome U133 Plus 2.0 GeneChip (Affymetrix, Santa Clara, Calif.) (42).

Analyses were performed using one patient sample per chip. Image files were captured using an Affymetrix GeneChip Scanner 3000. .CEL files were subsequently preprocessed using Robust Multiple-array Average (RMA) normalization (21) using GeneSpring GX 7.3 software (Agilent Technologies, Palo Alto, Calif.). All signal intensity-based data was used after RNA normalization, which specifically suppresses all but significant variation among lower intensity probe sets (21). All chips were then normalized to the respective median values of controls. Differences in mRNA abundance between patient samples were determined using GeneSpring GX 7.3. All statistical analyses used corrections for multiple comparisons. The specific statistical and filtering approaches are provided in the Results section because of their relevance to data interpretation.

Two-dimensional cluster maps were constructed using GeneSpring GX 7.3. Gene trees are represented in the vertical dimension and condition trees are represented in the horizontal dimension. Both the gene trees and condition trees are based on the Pearson similarity algorithm. The coloring conventions for all maps are as follows: red intensity correlates with increased gene expression, blue intensity correlates with decreased gene expression, and yellow intensity correlates with no change in gene expression relative to the median of controls.

Ontology analyses were performed by uploading specific gene expression lists to the web-based application D.A.V.I.D. (Database for Annotation, Visualization and Integrated Discovery) that allows public access to a relational database of functional gene annotations (13) Canonical pathway analyses were performed by uploading specific gene lists to the Ingenuity Systems Pathways Knowledge Base (Ingenuity Systems, Redwood City, Calif.) that provides a tool for discovery of canonical pathways within the uploaded gene lists (6). Both applications use specific approaches to estimate significance based on non-redundant representations of the microarray chip and to convert the uploaded gene lists to gene lists containing a single value for each gene.

Ancillary validation studies: Real time quantitative PCR was performed for selected genes by a standard approach involving the Superscript First Strand Synthesis kit (Invitrogen, Carlsbad, Calif.), SYBR green (BioRad, Hercules, Calif.) and the iCycler Thermal Cycler (BioRad). Serum interleukin-8 was measured using an ELISA kit, as specified by the manufacturer (Biosource, Camarillo, Calif.).

### Results

General study subject data: Summary demographic, clinical laboratory, and microbiologic data for the study subjects are provided in Tables 1 and 2. A total of 57 individual microarray chips, representing 15 individual controls and 42 individual patients with septic shock, were used for analysis. The controls were comparable to the patients with septic shock with regard to age, race, and gender. Among the patients with septic shock there were 28 having positive identification of an infecting organism (67%) and 9 deaths (21% mortality). Overall, the patients with septic shock were heterogeneous with respect to age, illness severity, gender, race, infectious organisms, and sites of infection. The annotated clinical data provided in Table 1 were not significantly different between septic shock survivors and septic shock nonsurvivors, except that the nonsurvivors had a significantly higher (p<0.05) severity of illness score (PRISM score) than the survivors.

Differential gene expression in pediatric septic shock: To begin testing the hypothesis that pediatric septic shock is characterized by broad alterations in gene expression, we conducted a two group ANOVA (Benjamini-Hochberg false discovery rate of 5%) using controls and patients with septic shock as the comparison groups, and all gene probes within the microarray (54,681 gene probes). This statistical filter yielded a working list of 17,601 gene probes that were differentially regulated between controls and patients with septic shock. To further refine this 17,601 gene list, we next applied an expression filter that selected only the genes, within the above 17,601 gene list, having at least 2-fold expression difference in at least 50% of the patients with septic shock, compared to the median of the controls. This expression filter yielded a final working list of 2,482 gene probes that were differentially regulated between patients with septic shock and controls.

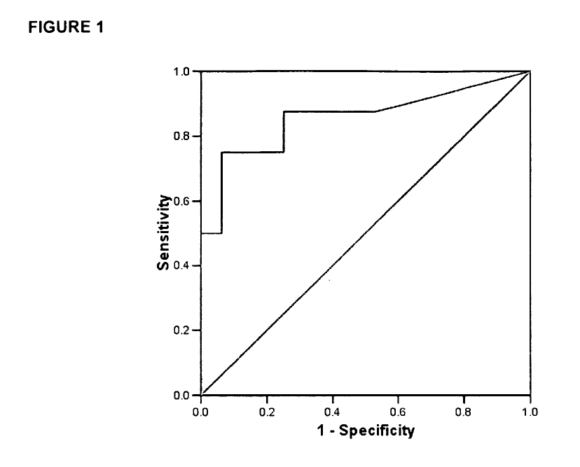

These 2,482 gene probes were then subjected to two-dimensional cluster analysis depicted in FIG. 1 All of the patients with septic shock cluster together at the center of the map in a homogenous manner, thus demonstrating a relative commonality of gene regulation on Day 1 of pediatric septic shock. This homogenous clustering is dependent on a group of genes in the upper portion of the map having increased expression (1,081 genes) and a group of genes in the lower portion of the map having decreased expression (1,401 genes).

In order to begin deriving biological meaning from these 2,482 differentially regulated genes, we uploaded the individual list of genes with increased expression and the individual list of genes with decreased expression to both the D.A.V.I.D. database (13) and the Ingenuity Systems Pathways Knowledge Base (6). As shown in Tables 3 and 4, the D.A.V.I.D.-dependent analyses yielded several relevant functional annotations within both gene lists. The functional annotations derived from the list of genes with increased expression (Table 3) are consistent with the current literature focused on septic shock (3, 34). The functional annotations derived from the list of genes with decreased expression (Table 4) are notable for the prevalence of zinc- and metal binding-related ontologies.

As shown in Tables 5 and 6, the Ingenuity Systems Pathways Knowledge Base-dependent analysis also yielded several relevant canonical pathways within both gene lists. Similar to the functional annotations listed in Table 3, the majority of canonical pathways derived from these gene lists are consistent with the current experimental literature focused on septic shock (3, 34)

### Differential Gene Expression Between Survivors and Nonsurvivors of Pediatric Septic Shock:

Since our initial attempt at elucidating the genome level response of children with septic shock yielded biologically plausible data, we next tested the hypothesis that there is a differential pattern of gene expression between survivors and nonsurvivors of pediatric septic shock. To this end, we conducted a three group ANOVA (Benjamini-Hochberg false discovery rate of 5%) using controls, septic shock survivors, and septic shock nonsurvivors as the comparison groups, and all gene probes within the microarray (54,681 gene probes). This statistical filter yielded a working list of 13,054 gene probes that were differentially regulated between the three groups. A post hoc Tukey test indicated that 589 of these 13,054 gene probes were differentially regulated between the survivors and the nonsurvivors. To further refine this gene list, we applied an expression filter that selected only the genes, within the above 589 gene list, having at least 2-fold expression difference in at least 50% of the survivors, compared to the median of the nonsurvivors. This expression filter yielded a final working list of 63 gene probes that were differentially regulated between survivors and nonsurvivors.

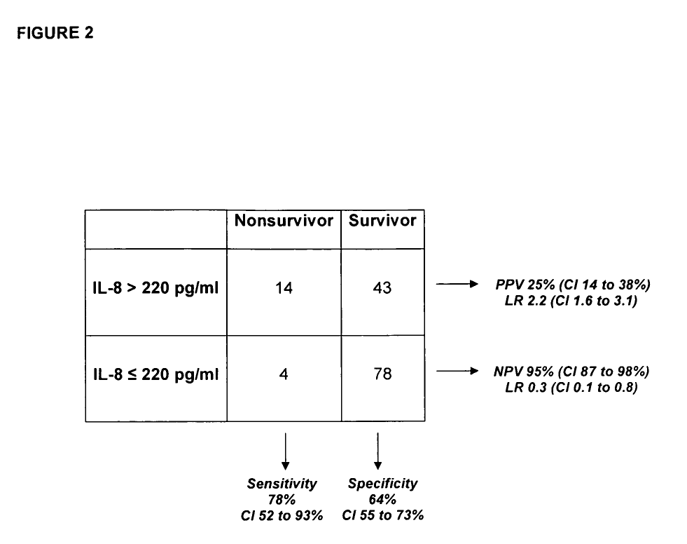

These 63 gene probes were then subjected to two-dimensional cluster analysis as depicted in FIG. 2. All of the nonsurvivors cluster to the left side of the map, thus demonstrating a relative commonality of gene regulation in nonsurvivors and survivors, respectively. As shown in Table 7, D.A.V.I.D.-based analysis of the 63 gene probes depicted in FIG. 2 yielded several biologically relevant functional annotations, including the “metal-binding” functional annotation previously noted in Table 4. Among the 63 gene probes depicted in FIG. 2, 36 gene probes (corresponding to 34 individual genes, Table 8) were upregulated and 27 gene probes (corresponding to 26 individual genes, Table 9) were downregulated in the nonsurvivors relative to survivors, respectively. The two gene lists provided in Tables 8 and 9 represent potential biomarkers for poor outcome and/or potential novel therapeutic targets in the context of pediatric septic shock.

### Validation of Interleukin-8 Microarray Data:

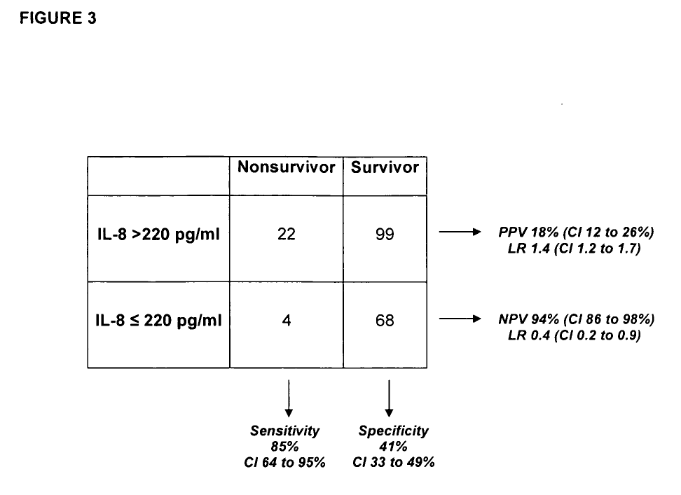

Our initial approach to validating our microarray data involved two complementary approaches: expression validation and functional validation. Table 8 indicates that interleukin-8 (IL-8) mRNA expression is increased in nonsurvivors of pediatric septic shock, compared to survivors. Since IL-8 protein is readily detectable in serum samples, we assayed parallel serum samples from the same patient cohort by way of ELISA. As shown in FIG. 3, nonsurvivors had increased serum levels of IL-8 protein compared to survivors, thereby corroborating the IL-8-specific microarray data.

### DISCUSSION

These data represent the largest reported cohort of patients with septic shock (pediatric or adult), to date, which has undergone genome-level expression profiling based on microarray technology. The patients in this cohort are heterogeneous at several levels and received clinical care at multiple centers. Despite this heterogeneity and the potential confounding variables, the data are coherent and biologically plausible.

The data demonstrate that Day 1 of pediatric septic shock is characterized by broad alterations of gene expression and that these broad alterations can be identified through genome-level expression profiles generated from accessible, clinically relevant biological samples. Importantly, the coordinately regulated genes fit well within biologically relevant gene ontologies and canonical pathways. The gene ontologies (Table 3) and canonical pathways (Table 5) detected within the 1,081 upregulated genes in this patient cohort do not necessarily represent novel concepts. The existing experimental and clinical literature has well established that inflammation, immunity, and stress response-related genes are highly regulated in the context of septic shock (3, 34). Nevertheless, the current demonstration of these ontologies and pathways provide confidence that the overall data are biologically relevant, rather than being artifacts of this high throughput approach.

We are ultimately interested in determining whether or not there exist biologically significant gene expression profiles that distinguish survivors and nonsurvivors of pediatric septic shock. The rationale for addressing this question is to discover novel biomarkers of poor outcome and novel therapeutic targets as means for developing more effective therapeutic strategies. The current data represent an initial approach to this question given the relatively small number of nonsurvivors in the current patient cohort. The data demonstrate, however, that our microarray-based approach is a feasible means of addressing this question. Furthermore, the validity of these data is suggested by the demonstration that increased IL-8 mRNA expression in nonsurvivors (microarray) correlated with increased IL-8 protein levels (ELISA).

Within the current patient cohort, we have elucidated a relatively small group of genes that are differentially regulated between survivors and nonsurvivors of pediatric septic shock. The veracity of this particular gene expression profile to effectively predict survival versus nonsurvival is the subject of ongoing studies in which the current data set will serve as a learning data set and a separate future group of patients will serve as a validation data set. Nevertheless, the current data provide a foundation to begin formulating testable, novel hypotheses regarding the pathophysiology of poor outcome in pediatric septic shock, which may ultimately transfer to adult septic shock.

### REFERENCES

1 Abraham E. Why immunomodulatory therapies have not worked in sepsis. Intensive Care /Wee/25: 556-566, 1999.
2. Anonymous. FDA approves first biologic treatment for sepsis. FDA Consum 36:3, 2002.
3. Ayala A and Chaudry I H. Immune dysfunction in murine polymicrobial sepsis:mediators, macrophages, lymphocytes and apoptosis. ShockG Suppl 1. S27-38, 1996.
4. Bernard G R, Vincent J L, Laterre P F, La Rosa S P, Dhainaut J F, Lopez-Rodriguez A, Steingrub J S, Garber G E, Helterbrand J D, Ely E W, and Fisher C J, Jr. Efficacy and safety of recombinant human activated protein C for severe sepsis. N Engl J Med 344: 699-709, 2001.
5. Bhutta Z A, Nizami S Q, and Isani Z. Zinc supplementation in malnourished children with persistent diarrhea in Pakistan. Pediatrics 103: e42, 1999.
6. Calvano S E, Xiao W, Richards D R, Fciciano R M, Baker H V, Cho R J, Chen R O, Brownstein B H, Cobb J P, Tschoeke S K, Miller-Graziano C, Moldawer L L, Mindrinos M M, Davis R W, Tompkins R G, and Lowry S F. A network-based analysis of systemic inflammation in humans. Nature 437: 1032-1037, 2005.
7. Carcillo J A and Fields Al. Clinical practice parameters for hemodynamic support of pediatric and neonatal patients in septic shock. CritCare Mec/30: 1365-1378, 2002.
8. Chinnaiyan A M, Huber-Lang M, Kumar-Sinha C, Barrette T R, Shankar-Sinha
S, Sarma V J, Padgaonkar V A, and Ward P A. Molecular signatures of sepsis: multiorgan gene expression profiles of systemic inflammation. Am J Pathol 159: 1199-1209, 2001.
9. Cobb J P, Mindrinos M N, Miller-Graziano C, Calvano S E, Baker H V, Xiao W, Laudanski K, Brownstein B H, Elson C M, Hayden D L, Herndon D N, Lowry S F, Maier R V, Schoenfeld D A, Moldawer L L, Davis R W, Tompkins R G, Bankey P, Billiar T, Camp D, Chaudry I, Freeman B, Gamelli R, Gibran N, Harbrecht B, Heagy, W, Heimbach D, Morton J, Hunt J, Lederer J, Mannick J, McKinley B, Minei J, Moore E, Moore F, Munlord R, Nathens A, O'Keefe G, Purdue G, Rahme L, Remick, D, Sailors M, Shapiro M, Silver G, Smith R, Stephanopoulos G, Stormo G, Toner, M, Warren S, West M, Wolfe S, and Young V. Application of genome-wide expression analysis to human health and disease, Proc Natl Acad Sci USA 102: 4801-4806, 2005.
10. Coyle P, Philcox J C, Carey L C, and Rofe A M. Metallothionein: the multipurpose protein. Cell Mol Life Sci 59: 627-647, 2002.
11. Dave S S, Wright G, Tan B, Rosenwald A, Gascoyne R D, Chan W C, Fisher Rl, Braziel R M, Rimsza L M, Grogan T M, Miller T P, LeBlanc M, Greiner T C, Weisenburger D D, Lynch J C, Vose J, Armitage J O, Smeland E B, Kvaloy S, Holte, H, Delabie J, Connors J M, Lansdorp P M, Ouyang Q, Lister T A, Davies A T, Norton, A T, Muller-Hermelink H K, Ott G, Campo E, Montserrat E, Wilson W H, Jaffe E S, Simon R, Yang L, Powell J, Zhao H, Goldschmidt N, Chiorazzi M, and Staudt L M. Prediction of survival in follicular lymphoma based on molecular features of tumor-infiltrating immune cells. N Engl J Med 351•2159-2169, 2004.
12. Dellinger R P, Carlet T M, Masur H, Gerlach H, Calandra T, Cohen J, Gea-Banacloche J, Keh D, Marshall J C, Parker M M, Ramsay G, Zimmerman J L, Vincent J L, and Levy M M. Surviving Sepsis Campaign guidelines for management of severe sepsis and septic shock. Crit. Care Med 32: 858-873, 2004.
13. Dennis G, Jr., Sherman B T, Hosack D A, Yang J, Gao W, Lane H C, and Lempicki R A. DAVID: Database for Annotation, Visualization, and Integrated Discovery. Genome Biol 4\ P3, 2003.
14. Eisenberg P. Discontinuation of Study F1K-MC-EVBP, Investigation of the Efficacy and Safety of Drotrecogin Alfa (Activated) in Pediatric Severe Sepsis. Available at http://www.fda.gov/medwatch/SAFETY/2005/xigris dearHCP_{—}4-21-05.htm. Accessed Mar. 7, 2006.
15. Failla M L. Trace elements and host defense: recent advances and continuing challenges. J/vitfr 133: 1443S-1447S, 2003.
16. Fraker P J and King L E. Reprogramming of the immune system during zinc deficiency. Annu Rev Nutr 24: 277-298, 2004.
17. Gaetke L M, McClain C J, Taiwalkar R T, and Shedlofsky S I. Effects of endotoxin on zinc metabolism in human volunteers. Am J Physio!272: E952-956, 1997.
18. Goldstein B, Giroir B, and Randolph A. International pediatric sepsis consensus conference: definitions for sepsis and organ dysfunction in pediatrics. Pediatr Crit. Care Med 6: 2-8, 2005.
19. Hotz C, Peerson J M, and Brown K H. Suggested lower cutoffs of serum zinc concentrations for assessing zinc status: reanalysis of the second National Health and Nutrition Examination Survey data (1976-1980). Am J Clin Nutr 78: 756-764, 2003.
20. Ibs K H and Rink L. Zinc-altered immune function. J Nutr'ltt: 1452S-1456S, 2003.
21. Irizarry R A, Hobbs B, Collin F, Beazer-Barclay Y D, Antonellis K J, Scherf U, and Speed T P. Exploration, normalization, and summaries of high density oligonucleotide array probe level data. Biostatistics 4: 249-264, 2003.
22. King J C, Shames D M, and Woodhouse L R. Zinc homeostasis in humans. J A/ufr130: 1360S-1366S, 2000.
23. King L E, Osati-Ashtiani F, and Fraker P J. Apoptosis plays a distinct role in the loss of precursor lymphocytes during zinc deficiency in mice. J Nutr 132: 974 979, 2002.
24. Kitamura H, Morikawa H, Kamon H, Iguchi M, Hojyo S, Fukada T, Yamashita, S, Kaisho T, Akira S, Murakami M, and Hirano T. Toll-like receptor-mediated regulation of zinc homeostasis influences dendritic cell function. Nat Immunol 7: 971-977, 2006.
25. Liuzzi J P, Lichten L A, Rivera S, Blanchard R K, Aydemir T B, Knutson M D, Ganz T, and Cousins R J. Interleukin-6 regulates the zinc transporter Zip 14 in liver and contributes to the hypozincemia of the acute-phase response. Proc Natl Acad Sci USA 102:6843-6848, 2005.
26. Pachot A, Lepape A, Vey S, Bienvenu I, Mougin B, and Monneret G. Systemic transcriptional analysis in survivor and nonsurvivor septic shock patients: a preliminary study. Immunol Leff 106: 63-71, 2006.
27. Pollack M M, Patel K M, and Ruttimann U E. The Pediatric Risk of Mortality Ill-Acute Physiology Score (PRISM III-APS): a method of assessing physiologic instability for pediatric intensive care unit patients. J Pediatrics: 575-581, 1997
28. Proulx F, Fayon M, Farrell C A, Lacroix J, and Gauthier M. Epidemiology of sepsis and multiple organ dysfunction syndrome in children. Chest 109: 1033-1037, 1996.
29. Prucha M, Ruryk A, Boriss H, Moller E, Zazula R, Herold I, Glaus R A, Reinhart K A, Deigner P, and Russwurm S. Expression profiling: toward an application in sepsis diagnostics. Shock 22: 29-33, 2004.
30. Rink L and Haase H. Zinc homeostasis and immunity. Trends Immunol 28: 1-4, 2007.
31 Rosenwald A, Wright G, Chan W C, Connors J M, Campo E, Fisher Rl, Gascoyne R D, Muller-Hermelink H K, Smeland E B, Giltnane J M, Hit E M, Zhao H, Averett L, Yang L, Wilson W H, Jaffe E S, Simon R, Klausner R D, Powell J, Duffey P L, Longo D L, Greiner T C, Weisenburger D D, Sanger W G, Dave B J, Lynch J C, Vose J, Armitage J O, Montserrat E, Lopez-GuiMerino A, Grogan T M, Miller T P, LeBlanc M, Ott G, Kvaloy S, Delabie J, Holte H, Krajci P, Stokke T, and Staudt L M. The use of molecular profiling to predict survival after chemotherapy for diffuse large-B-cell lymphoma. N Engl J Med 346:1937-1947, 2002.
32. Ruel M T, Rivera J A, Santizo M C, Lonnerdal B, and Brown K H. Impact of zinc supplementation on morbidity from diarrhea and respiratory infections among rural Guatemalan children Pediatrics 99: 808-813, 1997.
33. Shankar A H and Prasad A S. Zinc and immune function: the biological basis of altered resistance to infection. Am J Clin Nutr 68: 447S-463S, 1998.
34. Shanley T P, Hallstrom C, and Wong H R. Sepsis. In Pediatric Critical Care Medicine, 3rd ed. B. P Fuhrman and J. J. Zimmerman, eds. St. Louis, Mo.: Mosby, 1474-1493, 2006.
35. Sheehan M, Wong H R, Hake P W, and Zingarelli B. Parthenolide improves systemic hemodynamics and decreases tissue leukosequestration in rats with polymicrobial sepsis. CritCare Mec/31′ 2263 2270, 2003.
36. van de Vijver M I, He Y D, van't Veer L J, Dai H, Hart A A, Voskuil D W, Schreiber G J, Peterse J L, Roberts C, Marton M J, Parrish M, Atsma D, Witteveen A, Glas A, Delahaye L, van der Velde T, Bartelink H, Rodenhuis S, Rutgers E T, Friend S H, and Bernards R. A gene-expression signature as a predictor of survival in breast cancer. N Engl J Mec/347: 1999-2009, 2002.
37. von Bulow V, Rink L, and Haase H. Zinc-mediated inhibition of cyclic nucleotide phosphodiesterase activity and expression suppresses TNF-alpha and IL-1 beta production in monocytes by elevation of guanosine 3′,5′-cyclic monophosphate. J Immunol 175: 4697-4705, 2005.
38. Waelput W, Broekaert D, Vandekerckhove J, Brouckaert P, Tavernier J, and Libert C. A mediator role for metallothionein in tumor necrosis factor-induced lethal shock. J Exp Med 194: 1617-1624, 2001.
39. Watson R S, Carcillo J A, Linde-Zwirble W T, Clermont G, Lidicker J, and Angus D C. The epidemiology of severe sepsis in children in the United States. Am J Respir Crit. Care Med 167: 695-701, 2003.
42. Wilkinson J D, Pollack M M, Ruttimann U E, Glass N L, and Yeh T S. Outcome of pediatric patients with multiple organ system failure. Crit. Care Med 14: 271-274, 1986.
41 Zhou Z, Wang L, Song Z, Saari J T, McClain C J, and Kang Y J. Abrogation of nuclear factor-kappaB activation is involved in zinc inhibition of lipopolysaccharide-induced tumor necrosis factor-alpha production and liver injury. Am J Pathol′\64: 1547-1556, 2004.
42. Zimmermann N, King N E, Laporte J, Yang M, Mishra A, Pope S M, Muntel E E, Witte D P, Pegg A A, Foster P S, Hamid Q, and Rothenberg M E. Dissection of experimental asthma with DNA microarray analysis identifies arginase in asthma pathogenesis. J Clin Invest 111:1863-1874, 2003.

1. A method for the diagnosis and prognosis of sepsis in a mammalian subject comprising the following steps: (a) obtaining a biological sample from an individual suspected of having sepsis; (b) determining the expression level or protein concentration of IL-8 in the sample using a direct or an indirect detection technique; and (c) correlating the expression level or protein concentration of IL-8 in the sample to known standards.

2. The method according to claim 1, wherein the biological sample is selected from the group consisting of body fluid, tissue and cell lysate.

3. The method according to claim 2, wherein the body fluid is selected from the group consisting of blood, serum, urine, lymph, saliva, amniotic fluid, prostatic fluid, seminal fluid, biopsy fluid, gastrointestinal fluid, vaginal fluid and combinations thereof.

4. The method of claim 3, wherein the sepsis is a pathology selected from the group consisting of sepsis, sepsis progressing to septic shock, sepsis likely to progress to septic shock or septic shock.

5. The method of claim 4, wherein the sepsis is septic shock.

6. The method of claim 4, wherein the sepsis patient is 10 years of age or younger.

7. The method of claim 5, wherein IL-8 is detected using an agent selected from the group consisting of antibodies that bind IL-8, IL-8 binding partners, and nucleic acids that hybridize to a nucleic acid encoding IL-8.

8. The diagnostic method of claim 7, wherein the direct or indirect detection technique involves an immunoassay.

9. The method of claim 8, wherein the agent is tagged with a label.

10. The method of claim 9, wherein the label is a radioactive label, a fluorescent label, an enzyme, or a chemiluminescent tag.

11. The method of claim 8, wherein the immunoassay comprises immunoblotting, immunodiffusion, immunoelectrophoresis, or immunoprecipitation.

12. The method of claim 11, wherein IL-8 is detected by dot blotting.

13. The method of claim 12, wherein dot blotting comprises using a Bio-Dot SF module.

14. The method of claim 1, wherein IL-8 is detected by nucleic acid hybridization.

15. The method of claim 14, wherein the nucleic acid hybridization is RT-PCR or Northern blot analysis.

16. The method according to claim 5, further comprising correlating the binding of the biomarker in the sample to known standards to predict the severity of the septic shock.

17. The method according to claim 5, further comprising correlating the binding of the biomarker in the sample to known standards to predict whether the subject will require standard care or high risk treatment.

18. The method according to claim 5, further comprising the binding of the biomarker in the sample to known standards to select the appropriate therapeutic treatment for the septic shock, wherein elevated binding of the biomarker indicates that the patient is a candidate for therapies selected from antibiotics, organ support, or combinations thereof.

19. The method according to claim 17, wherein individuals having levels of IL-8 protein less than about 220 pg/ml, are considered appropriate candidates for standard care.

20. A method for the stratification of a sepsis condition in a mammalian subject for determining the effective course of treatment comprising the steps: (a) obtaining a biological sample from a subject suspected of having sepsis; (b) determining the expression level or protein concentration of IL-8 in the sample using a direct or an indirect detection technique; and (c) identifying the individual as appropriate for a particular treatment.

21. The method according to claim 20, wherein the biological sample is selected from the group consisting of body fluid, tissue and cell lysate.

22. The method according to claim 21, wherein the body fluid is selected from the group consisting of blood, serum, urine, lymph, saliva, amniotic fluid, prostatic fluid, seminal fluid, biopsy fluid, gastrointestinal fluid, vaginal fluid and combinations thereof.

23. The method of claim 22, wherein the sepsis is a pathology selected from the group consisting of sepsis, sepsis progressing to septic shock, sepsis likely to progress to septic shock or septic shock.

24. The method of claim 23, wherein the sepsis is septic shock.

25. The method according to claim 24, further comprising selecting the appropriate therapeutic treatment for the septic shock.

26. The method according to claim 25, wherein individuals having levels of IL-8 protein less than about 220 pg/ml, are considered appropriate candidates for standard care.

27. The method according to claim 25, wherein the course of treatment is high risk treatment comprising a treatment selected from the group consisting of plasmapheresis, high dose ultrafiltration and extracorporeal membrane oxygenation

28. The method of claim 24, wherein the sepsis patient is 10 years of age or younger.

29. The method of claim 24, wherein IL-8 is detected using an agent selected from the group consisting of antibodies that bind IL-8, IL-8 binding partners, and nucleic acids that hybridize to a nucleic acid encoding IL-8.

30. The diagnostic method of claim 24, wherein the direct or indirect detection technique involves an immunoassay.

31. The method of claim 30, wherein the agent is tagged with a label.

32. The method of claim 31, wherein the label is a radioactive label, a fluorescent label, an enzyme, or a chemiluminescent tag.

33. The method of claim 30, wherein the immunoassay comprises immunoblotting, immunodiffusion, immunoelectrophoresis, or immunoprecipitation.

34. The method of claim 20, wherein IL-8 is detected by nucleic acid hybridization.

35. The method of claim 34, wherein the nucleic acid hybridization is RT-PCR or Northern blot analysis.

36. The method according to claim 25, wherein subjects having IL-8 protein levels greater than about 1500 pg/ml are considered as candidates for active agents that have been identified as efficacious for the treatment of septic shock using IL-8 levels as a treatment criteria or for other higher risk therapies.

37. The method according to claim 25, wherein subjects having IL-8 protein levels greater than about 1000 pg/ml are considered as candidates for active agents that have been identified as efficacious for the treatment of septic shock in trials using IL-8 levels as a treatment criteria, or for other higher risk therapies.

38. The method according to claim 25, wherein subjects having IL-8 protein levels less than about 1000 pg/ml are considered as candidates for active agents that have been identified as efficacious for standard care treatment of septic shock.

39. The method according to claim 25, wherein subjects having IL-8 protein levels less than about 500 pg/ml are considered as candidates for active agents that have been identified as efficacious for standard care treatment of septic shock.

40. The method according to claim 25, wherein subjects having IL-8 protein levels less than about 300 pg/ml are considered as candidates for active agents that have been identified as efficacious for standard care treatment of septic shock.

41. The method according to claim 25, wherein subjects having IL-8 protein levels less than about 220 pg/ml are considered as candidates for active agents that have been identified as efficacious for standard care treatment of septic shock.

42. A method for the stratification of a sepsis condition in a mammalian subject for determining the effective course of treatment comprising the steps: (a) obtaining a biological sample from a subject suspected of having sepsis; (b) determining the expression level or protein concentration of IL-8 in the sample using a direct or an indirect detection technique; and (c) correlating the expression level or protein concentration of IL-8 in the sample to known standards as a biomarker in septic shock patients as an indicator of the likelihood of survival.

43. A method for the stratification of a sepsis condition in a mammalian subject for determining the effective course of treatment comprising the steps: (a) obtaining a biological sample from a subject suspected of having sepsis; (b) determining the expression level or protein concentration of IL-8 in the sample using a direct or an indirect detection technique; and (c) correlating the expression level or protein concentration of IL-8 in the sample to known standards as a biomarker in septic shock patients for the purposes of structuring, conducting, or evaluating clinical trials or data from clinical trials.

44. A method for the stratification of a sepsis condition in a mammalian subject for determining the effective course of treatment comprising the steps: (a) obtaining a biological sample from a subject suspected of having sepsis; (b) determining the expression level or protein concentration of IL-8 in the sample using a direct or an indirect detection technique; (c) comparing the determined IL-8 expression or protein level to a set of predetermined values for IL-8; and (d) categorizing the individual for purposes of structuring, conducting or evaluating the clinical trial.

45. The method according to claim 44, wherein the method is retroactively applied to data derived from a clinical data for septic shock, wherein patients are stratified for the purposes of restructuring or removing patient data.

46. The method according to claim 44, wherein the method comprises using IL-8 levels to define a patient's entry into or exclusion from a clinical trial; defining a particular patient as a control or test subject; conducting post-hoc evaluation of clinical trial data; or other uses related to structuring the clinical trial or resulting data.

47. A diagnostic kit for the diagnosis and prognosis of septic shock in a mammalian subject comprising: a probe specific for IL-8 wherein the probe is capable of detecting a concentration of anti-IL-8 antibodies, such that a diagnosis or prognosis of the subject may be made; and reactants for detecting the concentration of anti-IL-8 antibodies.

48. The diagnostic kit according to claim 47, wherein the reactants for detecting the concentration of anti-IL-8 antibodies function in a method selected from the group consisting of in situ hybridization, hybridization, and recognition by marked specific antibodies, the method being conducted on filter, on solid support, in solution, or on gel, by using at least one technique selected from the group consisting of a sandwich method, Dot blot hybridization, isotopic or non-isotopic labeling, cold probe techniques, double immunodiffusion, counter-immunoelectrophoresis, and hemagglutination.

49. The diagnostic kit according to claim 47, wherein the probe is an antigen reactive to anti-IL-8 antibodies.

50. The diagnostic kit according to claim 47, wherein the probe is immobilized on a solid support.

51. The diagnostic kit according to claim 47, wherein the antigen forms an antigen-antibody complex with the anti-IL-8 antibodies.

52. The diagnostic kit according to claim 47, wherein the detection reactants comprise a reporter group conjugated to a binding agent.

53. The diagnostic kit according to claim 47, wherein the probe is a phage particle expressing an antigen specific for anti-IL-8 antibody.

54. The method of claim 1, which further comprises, in (i), selecting at least one additional septic shock markers that increase or decrease in individuals with that risk factor relative to healthy individuals.

55. The method according to claim 54, wherein the septic shock marker is selected from the group consisting of: antioxidants, trace elements, indicators of septic shock, iron metabolism markers, homocysteine, enzymes having antioxidant functions, enzymes having pro-oxidant functions, enzymes for DNA repair, enzymes of the glutathione metabolism, stress proteins, proteins implied in apoptosis, transcription factors, cytokines and chemokines.

56. The method according to claim 55, wherein the antioxidant is selected from: vitamin A, vitamin C, vitamin E, reduced glutathione (GSH)/oxidized glutathione (GSSG), protein thiols, glutathione peroxidase and superoxide dismutase.

57. The method according to claim 56, wherein the transcription factor is selected from NfkappaB-a, c-Fos, C-jun, IkappaB-alpha, monoamine oxidase A, monoamine oxidase B, and peroxisome proliferative-activated receptor-alpha.

58. The method according to claim 57, wherein the cytokine or chemokine is selected from: IL-1, IL-2, IL-6, IL-1beta, IL-2 and TNF1 receptor associated protein.

59. The method according to claim 54, wherein the amount of the septic shock marker is determined by measuring the concentration of the oxidative marker.

60. The method according to claim 54, wherein the amount is determined by measuring the concentration of the gene transcript/mRNA encoding the oxidative marker.

61. The method according to claim 54, wherein the amount of at least two septic shock markers is determined in parallel.

62. The method according to claim 60, wherein the amount of septic shock marker is measured by using a DNA chip.

63. A process of detecting septic shock in a blood sample comprising cells, which method comprises: (i) extracting mRNA from cells from the blood sample, (ii) reverse transcribing the mRNA into cDNA, with labeling of the cDNA, and (iii) contacting the cDNA with a population of synthetic DNA fragments under hybridizing conditions, wherein the population of synthetic DNA fragments hybridizes with the cDNA when present due to gene expression under septic shock, and simultaneously detecting hybridization, whereupon septic shock in a blood sample comprising cells is detected.
